(19) United States
(12) Patent Application Publication
(10) Pub. No.: US 2019/0126096 A1
(43) Pub. Date: May 2, 2019

(54) APPARATUS FOR PROVIDING CONTROLLED FLOW OF INHALATION-AIR TO A USER

(71) Applicant: Mark Squibb, Bellvue, CO (US)

(72) Inventor: Mark Squibb, Bellvue, CO (US)

(21) Appl. No.: 15/798,195

(22) Filed: Oct. 30, 2017

(51) Int. Cl. A63B 23/18; A61M 16/10; A61M 16/20; A61M 11/04; A61M 16/06; A62B 18/02; A62B 25/00; A62B 18/10; A63B 24/00

(52) U.S. Cl. CPC A63B 23/18; A61M 16/107; A61M 16/204; A61M 11/041; A63B 24/0087; A62B 18/02; A62B 25/005; A62B 18/10; A61M 16/06

(57) ABSTRACT

The present invention offers various advantages as it allows switching of the position of the valve to allow release of selective inhalation-air from the air-reservoir. This principle is utilized in restoring the blood flow, accelerate tissue regeneration, improve physical performance, improve fluid intelligence, disease avoidance, disease recovery. The apparatus is useful for athletes to do altitude contrast training.

### CROSS REFERENCE TO RELATED PATENT APPLICATION

The present invention claims priority to the provisional patent application No. 61/974,699 filed on Apr. 3, 2014 and is a continuation-in-part of nonprovisional application Ser. No. 14/663,881 filed Mar. 20, 2015, all of which are incorporated herein by reference in their entireties.

### BACKGROUND OF THE INVENTION

The present invention generally relates to an apparatus for providing inhalation-air from multi-compartment air-reservoir to a mask, and more particularly relates to an apparatus for switching in between a high oxygen concentration and a contrasting low oxygen concentration to provide a preferred concentration of oxygen to a user. The high oxygen concentration and low oxygen concentration are maintained in separate chambers.

Events that reduce the dissolved oxygen concentration in blood plasma induce adverse changes in health status. These changes are richly documented in the book Oxygen Multistep Therapy, by Manfred von Ardenne, herein included by reference, with a specific discussion of a vascular inflammatory mechanism within the first chapter, Physiological Mechanisms.

Short-term reductions in blood plasma oxygen concentration often cause endothelial inflammation, to create persistent, and often permanent vascular constrictions. These reductions often follow chemical, physical or emotional stress events. These constrictions reduce blood and oxygen delivery to downstream tissue causing tissue distress, disease vulnerability and accelerated degeneration.

Long-term reduction in plasma oxygen deprives avascular cells structures, like cartilage, ligaments, white blood cells, and lens of the eye, of oxygen, resulting in reduced elasticity, performance and healing capacity of avascular structures, including the vascular system itself. These plasma hypoxia conditions remain unrecognized and hence an in-actionable cofactor in many disorders.

Prior art systems utilize single air mixture, with a fixed oxygen partial pressure to administer extra oxygen to the body. Ardenne disclosed multiple methods of administering extra oxygen during physical challenge to increase the oxygen partial pressure of the respiratory mixture by using a fixed oxygen partial pressure in continuous delivery flow.

Ardenne also disclosed use of physical challenge as, exercise, heat, or pharmaceutical adrenal analogue to simultaneously up-regulate respiratory turbulence, as heart rate, and respiratory tidal volume. Increased respiratory turbulence caused more oxygen to dissolve in blood plasma resulting in a collection of methods to treat a plurality of health conditions that occurred as a consequence of blood plasma hypoxia.

Ardenne disclosed a fixed rate of supplemental oxygen during exercise ranging from 2-3 liters per minute to 50 liters per minute for athletes. It is known that for able-bodied individuals elevated rates of supplemental oxygen prevent the body from achieving maximum respiratory turbulence, and hence less than maximum achievable dissolved plasma oxygen.

Prior art systems that supply a fixed amount of extra oxygen during exercise increases oxygenation to only about half of what is achievable with the invention. The fixed elevation in oxygen partial pressure caused a net decrease in respiratory turbulence because extra oxygen makes it easier for the mammal's heart and lungs to meet respiratory demand.

This reduction in respiratory turbulence limits tissue perfusions because maximum heart rate and maximum arterial dilation are required to deliver maximum pulse pressure to capillaries. Exemplary tissue perfusion reflects the force of blood pumped by the heart, and the ability of oxygen enriched plasma to squirt past narrowed vascular narrow areas resulting from endothelial inflammation or injury.

The exemplary performance of the invention occurs when the mammal achieves a novel respiratory status of simultaneous maximum pulse and maximum oxygen partial pressure. This state is specifically induced when the briskly exerting mammal switches from a respiratory challenge status, respiratory mixture with reduced oxygen partial pressure, to respiratory recovery status, with a mixture with maximum oxygen partial pressure.

The novel exemplary effect occurs when the exerting mammal achieves simultaneous maximums of respiratory turbulence while breathing a mixture of maximum oxygen partial pressure. This occurs just after the switch from low oxygen partial pressure to high oxygen partial pressure. These moments, while the exerting mammal experiences of maximum heartbeat, with elevated oxygen partial pressure, create optimal conditions for tissue oxygen perfusion unachievable by any known prior art system.

These maximums are indicated by novel simultaneous physiological maximums: maximum oxygen tidal volume, maximum pulse rate, maximum oxygen partial pressure in the respiratory mixture, maximum force of blood in the venous structure, hypoxia induced vasodilation, all serve to create maximum force of blood pressure at the capillary entry, and hence maximal tissue blood perfusion for the mammal. It should be obvious to the skilled in the art that these simultaneous maximum conditions are unachievable by any prior art system due to the usage of single air concentration.

The novel achievement of these maximums produce rapid physiological effects from improved blood flow to organ systems and muscles throughout the body measured with pharmacological tests including mental performance. Therefore there is a need of an apparatus that reproduce physiological improvements disclosed by Ardenne, normally occurring in 36 hours using oxygen multistep methods, in approximately 15 minutes or less while providing two different concentrations of oxygen.

Further, the apparatus should provide more intense and more cumulative physiological improvements than those disclosed with prior art systems. Further, the apparatus should increase the testing of human athletic capacity increases dramatically and rapidly.

Many prior art systems utilize varying rates of oxygen delivery, but do not disclose use of contrasting air mixtures. There are three classifications of prior art systems, Oxygen Multistep, which delivers a fixed increase in oxygen partial pressure during exercise; hyperbaric which delivers a fixed level increased oxygen partial pressure at rest to the whole body; and hypoxic training systems that deliver a reduced partial pressure of oxygen at rest or during exercise to induce durable adaptive change for improved general oxygen utilization.

The key to dealing with blood plasma oxygen deficiency is to utilize the body's adaptive response to progressively contrasting altitudes. There have been various attempts at providing portable chambers that simulates different altitude to show the effects of increased altitude, and/or to obtain some of the advantages of simulating different altitudes for, e.g., athletic training. It has been used to train athletes for the purpose of improved athletic performance, pre-acclimatization to altitude and/or physical wellness.

In hypoxic chambers and exercise systems, the occupant is subjected to lower oxygen partial pressure such as to simulate high altitudes. It is well known to expose an exerting mammal to hypoxic conditions utilizing a respiratory mixture with a reduced oxygen partial pressure. This exposure creates beneficial vascular conditions known to improve distal tissue oxygenation. The beneficial effect normally occurs when a mammal adapts hypoxic conditions, which causes hypoxic vasodilation, and other effects.

Simultaneous hypoxic vasodilation with exertion causes increased pulse pressure at the capillary that squirts more blood through capillaries than normal. This enhanced pulse pressure improves tissue perfusion. The challenge in hypoxic exertion however, is that the blood plasma contains less oxygen than normal due to the reduced oxygen in the respiratory mixture. This reduction generally prevents oxygen dissolved the blood plasma from acting as an endothelial anti-inflammatory, as disclosed by Ardenne, and may provoke additional inflammation.

It should be apparent to one skilled in the art that the exemplary aspect of the invention utilizes hypoxic conditions to establish the hypoxic vasodilation to establish maximum pulse pressure at the capillary, and then switches to a maximal oxygen partial pressure, to change from the reduced oxygen plasma oxygen partial pressure available with prior art hypoxic training systems, to an enhanced oxygen partial pressure by the increased oxygen partial pressure.

This switch condition creates exemplary and novel conditions at the distal tissue, which are unachievable by non-switching hypoxic training systems that solely utilize a reduced oxygen partial pressure, or even during the recovery process when the exerting mammal recovers from the hypoxic training by recovering to normal air. The exemplary aspect of the invention utilizes the vascular conditions created by hypoxic exertion, immediately followed by enhanced oxygen. It should be apparent to one skilled in the art that the invention is therefore novel with respect to all forms of hypoxic training systems, and chambers.

Another type of simulation system includes hyperbaric chambers and are used in the medical and sports industries. In essence, occupants of hyperbaric chambers undergo hyperbaric treatments in which they are subjected to relatively high oxygen partial pressures. Hyperbaric treatments are known, amongst other things, to enhance muscular recuperation and to increase dissolved oxygen levels in body fluids.

Conventional hyperbaric chambers are typically made of rigid materials capable of withstanding pressure differentials. Accordingly, hyperbaric treatments are not commonly accessible and are often only available to elite-level athletes and selected patients.

However, prior art portable chambers have some shortcomings relative to the invention. Hyperbaric sessions have a physically slow response time, normally requiring 40 or more hours of use to produce a clinically measurable result. With the invention, equivalent, and usually superior results are achieved normally within about 3 minutes for able bodied users.

Hyperbaric chambers require whole body pressurization which often causes inner ear discomfort with most users. Physical encapsulation also causes claustrophobia for many users. Medical grade hyperbaric chambers require materials that cause them to cost at least 20× the amount of the invention. Medical hyperbaric administration requires one or two trained operators for safe administration health challenged individuals in a medical or professional context. Therefore, there is need of an apparatus to provide an enhanced form of exercise which is safe and easy to use for anyone capable of virtually any form of stationary exercise and does not require an administrator and can be used safely at home.

Hence, despite ongoing developments in the field of hyperbaric chambers, hypoxic breathing systems, and fixed mixture exercise with oxygen systems, there remains a need for a respiratory delivery system to create optimal physiological conditions for maximum oxygen partial pressure in blood plasma, and consequently tissue oxygen perfusion. This combination provides exemplary mitigation capacity of health conditions relating to plasma hypoxia, and inhibited tissue oxygen perfusion, and hence provides novel capacity to overcome shortcomings of prior art portable chambers used for hyperbaric and/or hypoxic treatments. These systems do not utilize rapidly switchable contrasting oxygen partial pressures of the invention. It should be apparent to one skilled in the art that prior art systems do not alone, or any practical combination, create the novel vascular conditions of the invention.

Accordingly, it would be desirable to have a more cost effective apparatus for providing controlled flow of inhalation-air from an air-reservoir to a mask that could better simulate contrasting altitudes, and in particular, easily simulate both lower and higher altitudes than the current altitude of a person. Further, the apparatus should be portable and should be set up at any place.

### SUMMARY OF THE INVENTION

The present invention provides an apparatus for providing a controlled flow of inhalation-air from an air-reservoir to at least one mask, the air-reservoir having a first air chamber to store a first concentration of the inhalation-air, and a second air chamber to store a second concentration of the inhalation-air.

In one embodiment, the present invention provides an apparatus for providing controlled flow of inhalation air from at least one multi-compartment air-reservoir to two or more masks simultaneously. In such an embodiment, multiple users can each have their own mask, whereby each mask has its own set of ducts for utilization of the various implements of the apparatus.

It is another embodiment of the present invention to have a reservoir having multiple chambers, wherein each chamber may hold a different gas.

In a further embodiment of the present invention, the apparatus may employ different type of supplemental agent introduction means, such as hydrolysis, vaporization, nebulization, and ionization.

In a still further embodiment, the apparatus provides a difference in oxygen partial pressures between at least two chambers of the reservoir, ranging from a maximum oxygen concentration exceeding 42% to 95%, and a reduced oxygen concentration reduced at least 20% to 60% below normal oxygen partial pressure.

It is an object of the present invention to provide an apparatus having a control unit and a switch unit, the control unit switches the source of inhalation-air flowing from the reservoir to the mask to change from between high oxygen concentration to a low oxygen concentration in order to provide a contrasting oxygen partial pressure of the inhalation-air to a user of the apparatus.

The mechanism of the present invention enables a user to achieve a maximum pulse and respiratory challenge using low oxygen concentration, and then switch to a high oxygen concentration air to utilize respiratory inertia with enhanced oxygen level in order to achieve maximum plasma oxygen saturation, and maximum physically achievable tissue oxygen perfusion.

The control unit of the invention includes a housing to receive the inhalation-air from the air-reservoir, a plurality of ducts protruding from the housing to connect with the air-reservoir and with at least one mask. The first duct is configured with the first air chamber to supply the first concentration of inhalation-air to the housing.

The second duct configured with the second air chamber to supply the second concentration of inhalation-air to the housing, and a third duct to transfer the received inhalation-air by the housing from the air-reservoir to the mask. The control unit further includes at least one valve configured to control the flow of inhalation-air from the first duct and the second duct to the housing. In one embodiment, wherein multiple masks are connected to the apparatus, each mask possessing its own set of ducts and its own control unit.

The switch unit positions the valve to selectively open and close the first duct and the second duct for regulating the flow of inhalation-air from the air-reservoir to the housing.

The switch unit includes a cable to move the valve to selectively open and close the first duct and the second duct for regulating the flow of inhalation-air from the air-reservoir to the housing, and a mechanical switch having a first position to actuate the cable to set the position of the valve for receiving the inhalation-air from the first duct, and a second position to actuate the cable to set the position of the valve for receiving the inhalation-air from the second duct.

The switch unit further includes a cable to move the valve to selectively open and close the first duct and the second duct and an electrical switch having a first position to actuate the cable to set the position of the valve for receiving the inhalation-air from the first duct; and a second position to actuate the cable to set the position of the valve for receiving the inhalation-air from the second duct.

In yet a further embodiment, the present invention allows control of pressure through the inclusion of a regulator and flowmeter.

The apparatus further includes plurality of filter units attached to each duct to filter the inhalation-air passing to the user, or users.

Furthermore, the housing includes a first strip attached on right side of the second duct to maintain the position of the valve, a second strip in between the first duct and the second duct to maintain the position of the valve, a third strip attached on right side of the first duct to maintain the position of the valve.

Another object of the present invention is to provide an apparatus for altitude contrast training of a user. The apparatus includes an air-reservoir, a mask, a control unit, a switch unit and one or more tubular conduits. The air reservoir includes a first air chamber to store a first concentration of inhalation-air, a second air chamber to store a second concentration of inhalation-air and a seam to separate the high-concentration chamber from the low-concentration chamber.

A further object of the invention is to control the inhalation pressure of the breathing mixture delivered to the user through the mask. This control is achieved through various means to create variable pressure ranging from positive pressure to assist breathing and negative pressure to increase breathing challenge.

Positive pressure generally means to aid the breathing phase as supporting inhalation by increasing the pressure of breathing mixture delivered to the mask. Negative pressure generally means to decrease the pressure within the mask to resist airflow during inhalation to increase the mechanical challenge of inhalation.

Positive pressure increases comfort for individuals with compromised respiratory systems enabling them to feel more comfortable as the positive pressure makes them feel like it is easier to breathe until they gain the strength. Applicant notes that positive pressure generally decreases the performance of gas exchange in the lungs. Positive pressure enables compromised individuals to tolerate exercise sufficiently regain strength that eventually enables them to benefit from improved gas exchange that occurs with negative pressure.

Negative pressure is preferable for athletic individuals and with competent respiratory processes. Negative pressure means to increase airflow resistance, causing the user to work harder to pull the breathing mixture into the lungs.

Negative pressure as airflow resistance improves carbon dioxide exchange by increasing the vacuum in the lungs during inhale which creates a greater partial pressure differential removing a greater percentage of carbon dioxide from the blood in the lungs resulting in more effective respiration. It also strengthens breathing muscles.

The exemplary value of variable pressure enables compromised users to achieve progressive improvement in respiratory efficiency gain strength-starting aided by positive pressure eventually decreasing assistance, and finally utilizing resistance to achieve optimal respiratory performance.

The following application discloses various embodiments that achieve variable pressure in the invention.

This embodiment utilizes the pressure surplus to create the positive pressure that is transferred to user through ducting and control unit to the mask.

Prior embodiments utilized a seam that isolates the pressure in high oxygen from the low oxygen chambers in the reservoir. There was no pressure transfer across the seam.

This further embodiment uses a flexible membrane to separate the high oxygen chamber from the low oxygen chamber. This embodiment uses the membrane to equalize pressure both chambers resulting in positive pressure in both compartments of the reservoir.

This embodiment uses a three layer design where the first layer, second layer, and third layer are stacked. The encapsulated between the first and second layer comprises the high oxygen chamber. The volume encapsulated between the second and third layer comprises the low oxygen chamber.

Addition of a first pressure release valve in the low oxygen compartment limits the pressure in both compartments to the pressure release setting in the low oxygen compartment.

Addition of a second pressure release valve in the high oxygen compartment enables the oxygen compartment to be pressurized to any valve equal or higher than the pressure in the low oxygen compartment without venting oxygen.

This embodiment enables pressure in both the high oxygen and low oxygen compartments to be controlled to overcome breathing mixture resistance in the breathing apparatus comprised of ducts and mask, and to provide mild positive pressure to the user.

A further embodiment uses a variable constriction in the duct connecting the control unit to the mask as a means of creating negative pressure.

A further embodiment uses a reversible fan in the duct connecting the control unit to the mask. The direction of flow of the fan determines the creation of positive or negative pressure. When the fan blows toward the mask positive pressure assists breathing. When the fan is off, the resistance of pulling the breathing mixture through the fan blades creates a negative pressure. When the fan is on in a reverse direction, the negative resistance is increased as the user is forced to overcome the negative pressure created by the fan.

Applicant discloses that an exemplary embodiment utilizes various combinations of positive and negative pressure to create variable resistance. For example, use of the flexible membrane combined with a variable duct constriction enable resistance to vary from the positive pressure of the reservoir with the constriction fully open, to the maximum resistance permitted by the duct constriction.

It is apparent to one skilled in the art that any embodiment utilizing any combination of positive pressure or negative pressure herein disclosed would be apparent to one skilled in the art without deviating from the scope of the invention.

It is yet another object of this invention to provide ambient air as one of the sources of air for one of the chambers.

Applicant further discloses that variable oxygen concentration in low oxygen mixture enhances benefits of invention. Variable oxygen concentration defines the oxygen partial pressure in the low oxygen breathing mixture supplied to the user during hypoxic challenge.

The exemplary range of the low oxygen mixture varies from ambient atmospheric level, approximately 21%, down to approximately 8%, is approximately % of ambient atmospheric levels. This range of variability enables exemplary performance of the invention across wide range of individuals with differing respiratory competency, ranging from severely challenged individuals to elite athletes.

Applicant further discloses two distinct and novel means for modulating the partial pressure of the low oxygen breathing mixture. Ranges from approximately 16% and 21% are achieved by limited air separation. Limited air separation is achieved in air separator units utilizing a variable flow oxygen valve that limits the amount of oxygen removed from filtrate air. Ranges between approximately 8% and 16% are achieved by selective release of higher oxygen filtrate during the partial swing absorption cycle hereinafter described.

Combined use of these means enables the invention to vary the low oxygen concentration between approximately 8% and 21% creating an exemplary challenge level for all users.

Health challenged users often prefer to limit the challenge level by utilizing a low oxygen mixture. As health challenged users become stronger they generally prefer increase the challenge level by reducing the oxygen concentration of the low oxygen mixture.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 illustrates a schematic diagram of an apparatus for providing controlled flow of inhalation-air, in accordance with a preferred embodiment of the present invention;

FIG. 2A illustrates the schematic diagram of an apparatus for providing controlled flow of the first concentration of inhalation-air from the first air chamber to the user, in accordance with a preferred embodiment of the present invention;

FIG. 2B illustrates the schematic diagram of an apparatus for providing controlled flow of the second concentration of inhalation-air from the second air chamber to the user, in accordance with a preferred embodiment of the present invention;

FIG. 3 illustrates a schematic diagram of an apparatus for providing controlled flow of inhalation-air from at least an air-reservoir to a mask, in accordance with another preferred embodiment of the present invention;

FIG. 4 illustrates the schematic diagram of an apparatus, in accordance with a preferred embodiment of the present invention; and

FIG. 5 illustrates the schematic diagram of a mechanical switch, in accordance with a preferred embodiment of the present invention.

FIG. 6 illustrates an embodiment of the apparatus having at least two masks.

FIG. 7 illustrates the use of at least two air chambers in an embodiment of the apparatus.

FIG. 8 illustrates the use of a nebulizer in an embodiment of the apparatus.

FIG. 9 illustrates the use of a regulator and flowmeter in an embodiment of the apparatus.

FIG. 10A illustrates the use of a membrane to equalize pressure in an embodiment of the apparatus.

FIG. 10B illustrates the use of a membrane to equalize pressure in an embodiment of the apparatus.

FIG. 10C illustrates the use of a membrane to equalize pressure in an embodiment of the apparatus.

FIG. 11A illustrates the use of an air-flow constriction in an embodiment of the apparatus.

FIG. 11B illustrates the use of an air-flow constriction in an embodiment of the apparatus.

FIG. 12A illustrates the air separators including a variable flow oxygen adjustment that determines the amount of oxygen removed from the filtrate air used as a source low oxygen air in an embodiment of the apparatus.

FIG. 12B illustrates the air separators including a variable flow oxygen adjustment that determines the amount of oxygen removed from the filtrate air used as a source low oxygen air in an embodiment of the apparatus.

FIG. 12C illustrates the air separators including a variable flow oxygen adjustment that determines the amount of oxygen removed from the filtrate air used as a source low oxygen air in an embodiment of the apparatus.

FIG. 13 illustrates a graphical representation of the variable flow oxygen adjustment.

FIG. 14a illustrates a graphical representation of the oxygen filter.

FIG. 14b illustrates a graphical representation of the concentration of gases across the filter.

FIG. 14c illustrates a graphical representation of the air near the inlet released during the vent.

FIG. 15 illustrates the pressure controlled diversion oxygen vent in an embodiment of the apparatus.

FIG. 16 illustrates an alternative apparatus using gas compression as a means.

FIG. 17 illustrates the compressed air reservoir in an alternate embodiment of the apparatus.

FIG. 18 illustrates the compressed air reservoir in an alternate embodiment used for multi-users.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE DRAWING

While this technology is illustrated and described in a preferred embodiment, an apparatus for providing controlled flow of inhalation-air from at least an air-reservoir to a mask of a user may be produced in many different configurations, forms and materials. There is depicted in the drawings, and will herein be described in detail, as a preferred embodiment of the invention, with the understanding that the present disclosure is to be considered as an exemplification of the principles of the invention and the associated functional specifications for its construction and is not intended to limit the invention to the embodiment illustrated. Those skilled in the art will envision many other possible variations within the scope of the technology described herein.

Reference will now be made in detail to several embodiments of the invention which are illustrated in the accompanying drawings. Wherever feasible and convenient, the same reference numerals are used in the figures and the description to refer to the same or like parts. The drawings are in a simplified form and not to precise scale. For purposes of convenience and clarity only, directional terms, such as top, bottom, left, right, up, down, over, above, below, beneath, rear, and front may be used with respect to the accompanying drawings.

These and similar directional terms should not be strictly construed to limit the scope of the invention. In addition, words such as attached, affixed, coupled, connected and similar terms with their inflectional morphemes are used interchangeably, unless the difference is noted or made otherwise clear from the context. These words and expressions do not necessarily signify direct connections, but include connections through mediate components and devices.

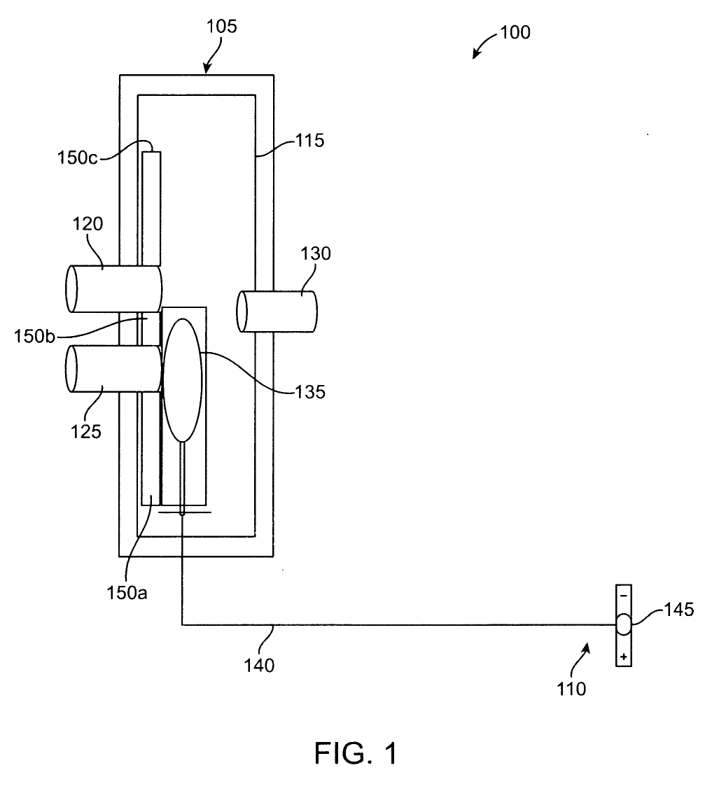

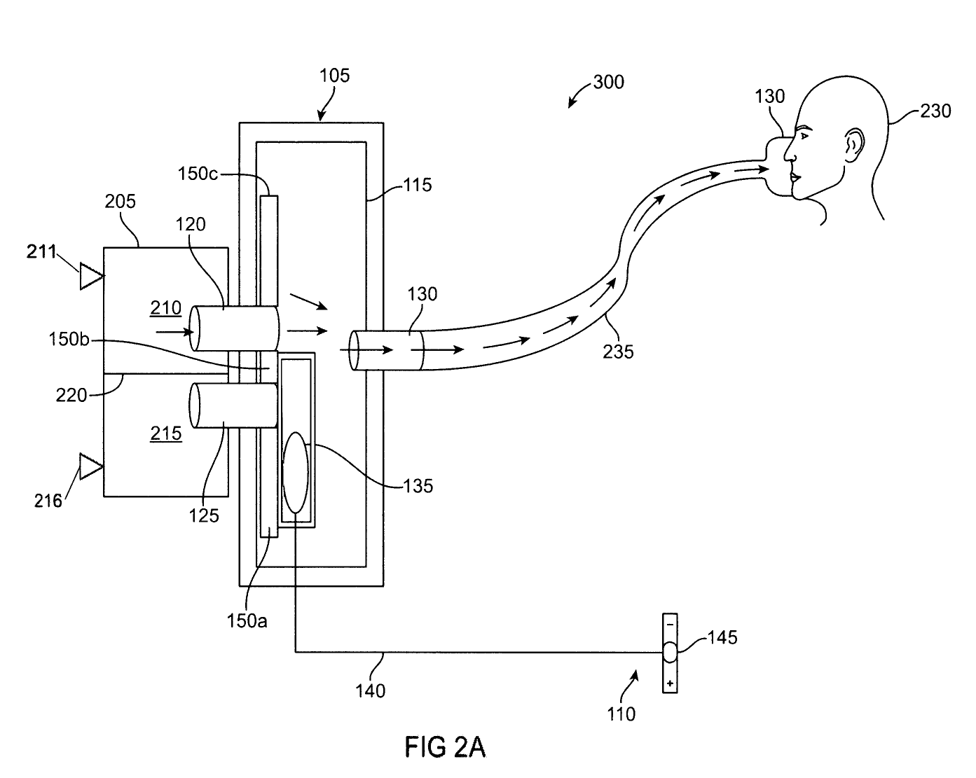

FIG. 1 illustrates valve embodiment of an apparatus 100 for providing controlled flow of inhalation-air from an air-reservoir (not shown in FIG. 1) in accordance with a preferred embodiment of the present invention. The air-reservoir having a plurality of air chambers, a first air chamber to store a first concentration of the inhalation-air, and a second air chamber to store a second concentration of the inhalation-air. In one embodiment, a compartment of an air reservoir may be the environment providing a source of gas with an ambient oxygen concentration. “Ambient” as used herein defined as the air existing in the environment surrounding the apparatus, wherein the ambient air is not connected in a chamber of the present invention. The air chambers are explained in detail in conjunction with FIG. 2 to FIG. 4 of the present invention.

The apparatus 100 includes a control unit 105 and a switch unit 110. The control unit 105 controls the flow of the inhalation-air. Further the control unit 105 receives air from the air-reservoir (not shown in FIG. 1) and transfers the inhalation-air to the user. The switch unit 110 positions the control unit 105 to selectively receive at least one of the inhalation-air from at least one of the chambers (not shown in FIG. 1) of the air-reservoir.

In a preferred embodiment of the present invention, the control unit 105 includes a housing 115 to receive the inhalation-air from the air-reservoir (not shown in FIG. 1), plurality of ducts such as a first duct 120, a second duct 125, and a third duct 225 protruding from the housing 115 to connect with the air-reservoir and with the user, and at least one valve 135 to control the flow of inhalation-air from the first duct 120 and the second duct 125 to the housing 115. The first duct 120 is configured with the first air chamber (explained in detail in conjunction with FIG. 2A) to supply the first concentration of inhalation-air to the housing 115.

The second duct 125 is configured with the second air chamber (explained in detail in conjunction with FIG. 2B) to supply the second concentration of inhalation-air to the housing 115. The third duct 225 is configured to transfer the received inhalation-air of the housing 115 to the mask (not shown in FIG. 1).

The valve 135 is configured to control the flow of inhalation-air from the first duct 120 and the second duct 125 to the housing 115. Examples of the valve 135 include but not limited to magnetic valves, air-actuated ball valves, and motorized ball valves, lead screw or linear actuator positioned flapper valves, or other valves configurations. It would be readily apparent to those skilled in the art that various types of valves 135 may also be envisioned to control the flow of inhalation-air without deviating from the scope of the invention.

The control unit 105 further includes at least one valve 135 configured to control the flow of inhalation-air from the first duct 120 and the second duct 125 to the housing 115. The third duct 125 transfers the inhalation-air from the housing 115 to at least one mask through a tubular conduit (explained in detail in conjunction with FIG. 2A, 2B, 3).

In one embodiment of the present invention, the control unit 105 switches the source of inhalation-air flowing from the air-reservoir to at least one mask to change from high oxygen concentration air to lower oxygen concentration air, in order to provide a contrasting oxygen partial pressure of the inhalation-air. The control unit enables the user to exert using a high respiratory challenge level to achieve maximum pulse and respiratory challenge under the low oxygen concentration air, and then switch to the high oxygen concentration air to utilize respiratory inertia with enhanced oxygen level to achieve maximum plasma oxygen saturation, and maximum physically achievable tissue oxygen perfusion.

In another embodiment, the control unit 105 has a switching mechanism that can provide adjustments so that the challenge level for user can be changed. “Challenge level” refers to the amount of oxygen available to a user of the system when using the system, the amount of oxygen being between a high oxygen concentration or a low oxygen concentration. A high-level challenge may be considered when the oxygen concentration is low, thus making it more difficult for the user to obtain sufficient oxygen. A low-level challenge may be considered when the oxygen concentration is high, thus making it easier for the user to obtain sufficient oxygen. The challenge level adjustment occurs by reducing the amount of oxygen in the air made available to the user.

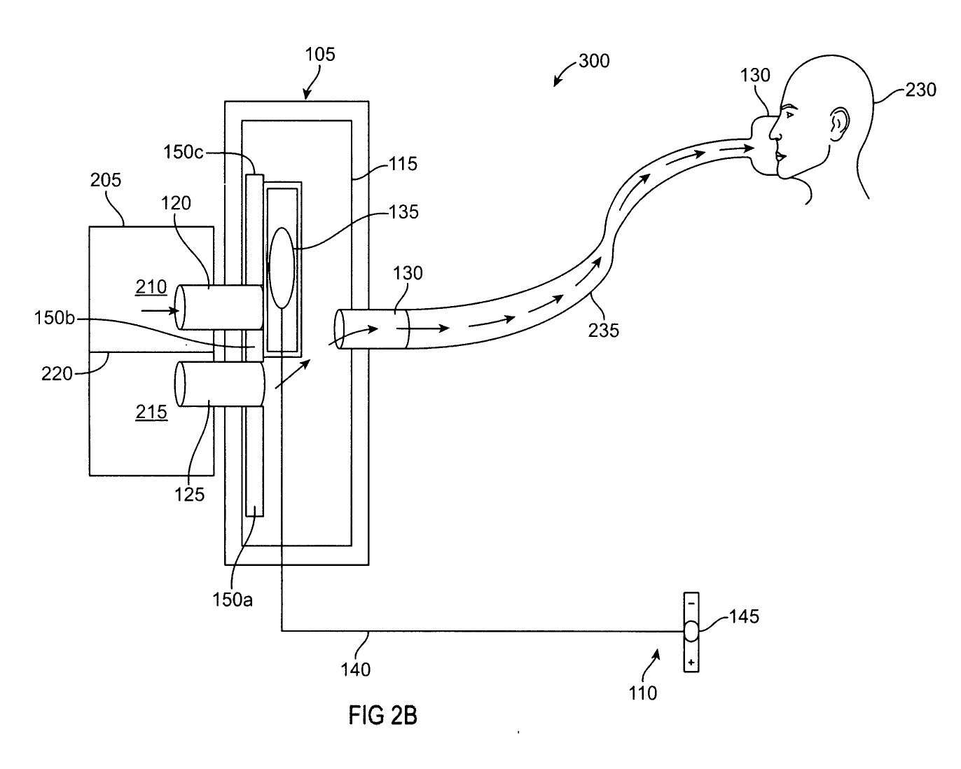

The apparatus 100 includes a switch unit 110 to position the valve 135 to selectively open and close the first duct 120 and the second duct 125 for regulating the flow of inhalation-air from the air-reservoir (not shown in FIG. 1) to the housing 115. The position of the valve 135 is explained in detail in conjunction with FIGS. 2A, 2B, and 3 of the present invention.

The switch unit 110 further includes a cable 140 and a mechanical switch 145. The cable 140 moves the valve 135 to selectively open and close the first duct 120 and the second duct 125 for regulating the flow of inhalation-air from the air-reservoir to the housing 115.

The mechanical switch 145 having a first position (explained in detail in conjunction with FIG. 2A) actuates the cable 140 to set the position of the valve 135 for receiving the inhalation-air from the first duct 120 and a second position (explained in detail in conjunction with FIG. 2B) to actuate the cable 140 to set the position of the valve 135 for receiving the inhalation-air from the second duct 125.

Examples of mechanical switch 145 includes but not limited to toggle switch, rocker switch, double pole switch, slide switch, rotary switch, key switch and tilt switch. It would be readily apparent to those skilled in the art that various type of the switch unit 110 may also be envisioned to switch the flow of inhalation-air without deviating from the scope of the invention. In a preferred embodiment of the present invention, the switch unit 110 may be operated mechanically by the user.

In an alternative embodiment, an underflow valve is included in the apparatus, which opens to the ambient air enabling the invention to automatically switch to ambient air when either compartment of the reservoir becomes empty.

In another embodiment of the present invention, the switch unit 110 may include a cable 140 and an electrical switch. The cable 140 moves the valve 135 to selectively open and close the first duct 120 and the second duct 125. The electrical switch may have a first position to actuate the cable 140 to set the position of the valve 135 for receiving the inhalation-air from the first duct 120 and a second position to actuate the cable 140 to set the position of the valve 135 for receiving the inhalation-air from the second duct 125.

Examples of electrical switch include but not limited to a motor in electrical connection with a source of electrical current and a direct current backup battery or other power storage device may be provided for positioning the valve 135.

In another embodiment of the present invention, the housing 115 includes a first strip 150a attached on right side of the second duct 12b to maintain the position of the valve 135, a second strip 150b in between the first duct 120 and the second duct 125 to maintain the position of the valve 135 and a third strip 150c attached on right side of the first duct 120 to maintain the position of the valve 135 with the housing 115.

Examples of the first strip 150a, second strip 150b and the third strip 150c includes but not limited to a magnetic strip, mechanical constraints or any other retaining units. However it would be readily apparent to those skilled in the art that various types of the strips may be used to maintain the position of the valve 135 without deviating from the scope of the invention.

FIG. 2A illustrates the schematic block diagram of an apparatus 200 for providing altitude contrast training to a user 230 in accordance with another embodiment of the present invention. The apparatus 200 includes an air-reservoir 205 to store inhalation-air, a mask 225, a control unit 105, a tubular conduit 235, and an adjustable airflow resistance unit.

The air-reservoir 205 includes a first air chamber 210 to store a first concentration of inhalation-air, a second air chamber 215 to store a second concentration of inhalation-air, and a seam 220 separating the first air chamber 210 from the second air chamber 215. The apparatus 200 may be particularly suited for use with an inhalation-air such as oxygen, nitrous oxide, medical air, carbon dioxide, helium, nitrogen, any other breathing gases etc. The first concentration of inhalation air is the high concentration inhalation-air and the second concentration of inhalation-air is the low concentration inhalation-air.

In one embodiment the air-reservoir 205 may include a physically separate first air chamber 210 and a second air chamber 215 to store a first concentration of inhalation-air and a second concentration of inhalation-air respectively. In another exemplary embodiment the air-reservoir 205 may have first air chamber 210 physically contained within the second air chamber 215.

In an embodiment of the present invention, the air-reservoir 205 is made of a flexible material that expands to store the inhalation-air. The inhalation-air is filled in the air-reservoir 205 by an external-air-source such as oxygen concentrator. The interior portion of the air-reservoir 205 is made of a medical grade or food grade membrane impervious to the contained inhalation-air (no plasticizers that give off chemicals) and the outer portion is made of durable, scuff resistant dust cover. However, it would be readily apparent those skilled in the art that various types of materials may be used to create air-reservoir 205 without deviating from the scope of the present invention.

Further, the air-reservoir 205 may be formed of a low-oxygen-permeability-material for accumulating the inhalation-air in an undiluted form. The air-reservoir 205 may be available in several sizes. Examples of the size of air-reservoir 205 may be around 1000 L capacity, 1500 L capacity etc. However, it would be readily apparent to those skilled in the art that various sizes of the air-reservoir 205 may be envisioned without deviating from the scope of the present invention. Typically, the air-reservoir 205 may be hung on the wall or any handy frame work nearby the work station.

In another embodiment, the difference in oxygen partial pressures between the chambers ranges from a higher (high) oxygen concentration exceeding 42% up to 95%, with a lower (low) oxygen concentration reduced at least 20% to 60% below normal oxygen partial pressure. However, it would be readily apparent to those skilled in the art that various concentrations of inhalation-air in the air-reservoir 205 may be envisioned without deviating from the scope of the present invention.

The mask 225 transfers the inhalation-air from the air-reservoir 205 to the user 230 for facilitating breathing. The mask 225 may be worn by the user 230 at the time of exercise e.g. cycling and may be made of plastic, silicone, or rubber. In a preferred embodiment of the present invention, the mask 225 may cover the nose and mouth (oral nasal mask) or the entire face (full-face mask) of the user 230.

The mask 225 may have a one way valve to breathe the inhalation-air in and may have a separate one way valve to breathe out into the atmosphere. However, it would be readily apparent to those skilled in the art various types of mask 225 such as nose cannula may be envisioned to deliver the inhalation-air to the user 230 without deviating from the scope of the invention.

The control unit 105 (explained in detail in conjunction with FIGS. A and 2B) controls the level of the inhalation-air flowing from the air-reservoir 205 to the mask 225 through the tubular conduits 235. The switch unit 110 is operated by the user 230 for positioning the valve 135 to selectively open and close the first duct 120 and the second duct 125 for regulating the flow of inhalation-air from the air-reservoir 205 to the housing 11b (explained in detail in conjunction with FIGS. 2A and 2B). However, it would be readily apparent to those skilled in the art that other users may also be able to operate the switch unit 110 without deviating from the scope of the present invention.

Further, the tubular conduit 235 allows the flow of inhalation-air from the housing 115 to the mask 225. The tubular conduit 235 may be of any dimension and may be made of plastic, silicone, or rubber. The tubular conduit 235 may be of several feet to allow the air-reservoir 205 to be positioned further away from the exercise equipment. Typically, the tubular conduit 235 delivers the oxygen in the range of 10-100 Liters per minute.

In another embodiment, the valve 135 may be operative to vary the ratio of the first concentration of inhalation air with the second concentration of inhalation air in such a way that the concentration of inhalation air in the housing 115 is in between the first concentration of inhalation-air and the second concentration of inhalation-air.

FIGS. 2A and 2B illustrates the schematic block diagrams of an apparatus 300 for providing controlled flow of the first concentration of inhalation-air from the first air chamber 210. FIG. 2B illustrates delivery of the second concentration of inhalation-air from the second air chamber 215 to the user 230 respectively, in accordance to one embodiment of the present invention.

In another embodiment of the present invention, the first concentration of inhalation-air is high concentration oxygen at or above 20.9% at sea level. Similarly, the second concentration of inhalation-air is low concentration oxygen at or below 20.9% at sea level. The low concentration oxygen is roughly equivalent to the amount of oxygen available at the high altitudes, but any oxygen concentration equal to or lower than ambient air is anticipated by the present invention.

In an exemplary embodiment as shown in FIG. 2B, the mechanical switch 145 is at a first position 146a for receiving the first concentration inhalation-air from the first air chamber 210 by the housing 115 through the first duct 120. The mechanical switch 145 pulls back the cable 140 to position the valve 135 against the second duct 125.

The valve 135 is attached to the first strip 150a and the second strip 150b and thus closes the path of the inhalation-air to flow through the second duct 125 from the second air chamber 215. Similarly as shown in FIG. 2B, the mechanical switch 145 is at a second position 146b for receiving the second concentration inhalation-air from the second air chamber 215 by the housing 115 through the second duct 125.

The mechanical switch 145 pushes the cable 140 to position the valve 135 against the first duct 120. The valve 135 is attached to the second strip 150b and third strip 150c and thus closes the path of the inhalation-air to flow through the first duct 120 from the first air chamber 210. Thus, the desired inhalation-air is then made to flow out of the housing 115 to the user 230 through the third duct 130 the tubular conduit 235 and the mask 225.

The aforementioned switching of high concentration of inhalation-air to low concentration of inhalation-air allows the user 230 to experience the physiological adaptations. It may help to restore two hormone cycles that fades with age i.e. erythropoietin (EPO) and human growth hormone (HGH). EPO triggers creation of red blood cells (RBC) which carry oxygen to the tissues.

Low concentration inhalation-air may cause hypoxic stress and may signal the body to increase EPO up to 1000 times to adapt to hypoxic challenge. HGH is an anabolic hormone that controls structural growth of bones and muscles. It is the main hormone of youth, and high levels are keys to both graceful aging and athletic performance. The apparatus 300 of the present invention helps the user in increasing HGH levels over 500%.

As shown in FIG. 3, an adjustable airflow resistance unit is included in the apparatus 236. Through such a mechanism, a positive resistance may be added during the transfer of inhalation air, meaning the user has to inhale harder than usual to get air, A negative resistance may be added during the transfer of inhalation air, which will make the air transfer easier. Through such mechanism, users, such as athletes, can utilize airflow constraint in the air-flow pathway to exercise their lungs. Health challenged users can adjust the pressure release valves in the high oxygen concentration and low oxygen concentration compartments to regulate positive pressure of the reservoir.

Suitable adjustable airflow resistance mechanisms can be selected from a fan inserted in the tubular conduit, and a regulator and a flowmeter; whereby the regulator is inserted on the tubular conduit.

In a further embodiment, ambient, or room, air can be used as a component in one of the air chambers. In such an embodiment, ambient air is pulled into the air chamber via a duct that is open, or the duct may be open to the atmosphere.

In another embodiment as shown in FIG. 3, the switch unit 110 includes a mechanical switch 145 which is at a neutral position. The valve 135 is attached to the second strip 150b and thus closes the path of the inhalation-air to flow through the first duct 120 and the second duct 125. Therefore, no inhalation-air is flowing from the air-reservoir 205 to the housing 115.

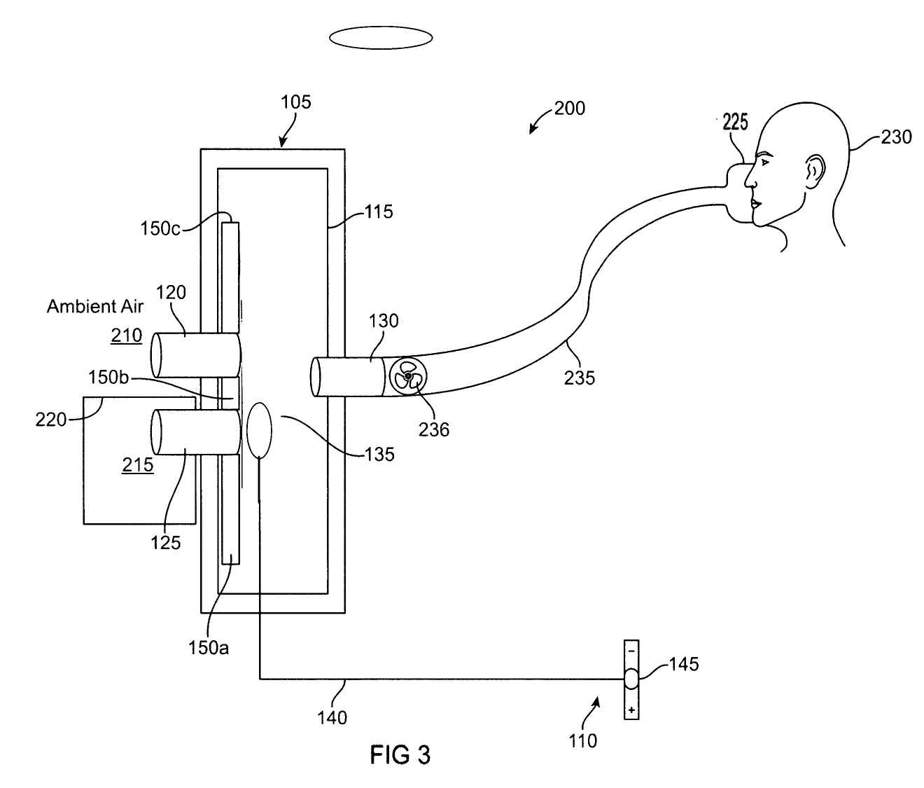

FIG. 4 illustrates the schematic block diagrams of an apparatus 400 for showing filter units 402. The apparatus 400 includes plurality of filter units 402 such as a first filter unit 402a and a second filter unit 402b attached to the first duct 120 and the second 125 respectively. The filter units 402 transfers the filtered inhalation-air received from the air-reservoir 205 to the housing 115. The filtered air is then transferred to the user 230 from the housing 115.

The filter units 402 may remove unwanted particulates from the inhalation-air such as airborne molecular contaminants etc. Examples of the filter unit 402 include but not limited to a cassette filter having sides of wire net, paper, carbon, foam, or cotton filters and spun fiberglass filter. The inhalation-air that is passed through the plurality of filter units 402 may pass through the filter textile from the air-reservoir 205 into the housing 115.

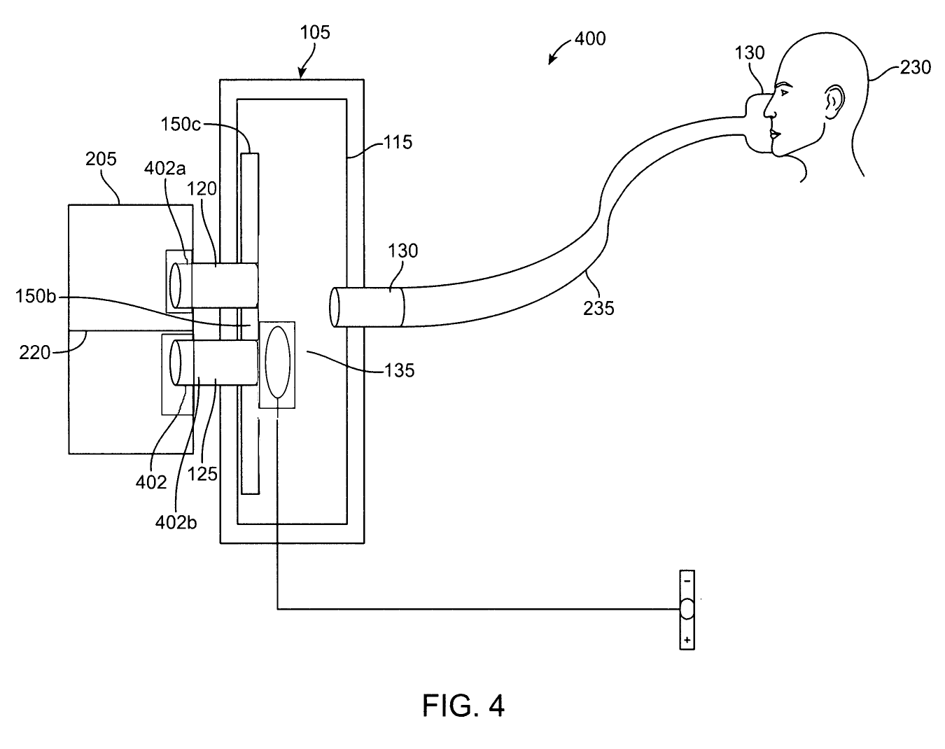

FIG. 5 illustrates the schematic diagram of a mechanical switch 145. The mechanical switch 145 includes a slide button 505. The slide button 505 is moved linearly to and from, to set the first position 146a i.e. +02 and the second position 146b i.e. −02 respectively and actuates the cable (not shown in fig.) to set the position of the valve for receiving the inhalation-air from the first duct or from the second duct, (explained in detail in conjunction with FIG. 3A and FIG. 3B respectively).

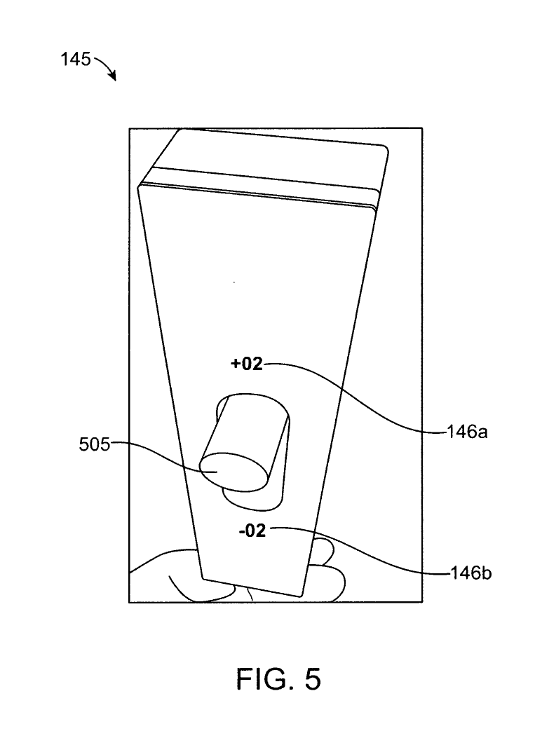

FIG. 6 is an embodiment of the apparatus 600 using two or more masks 635. In such an embodiment, several users 630 can use the apparatus 600 at the same time. Each user 630 uses their own mask to inhale oxygen or other gases. Each mask 635 has their own duct connected to the apparatus 600.

In yet a further embodiment, as shown in FIG. 6, the application 200 may include 2 or more mask, allowing multiple users to use the apparatus at the same time.

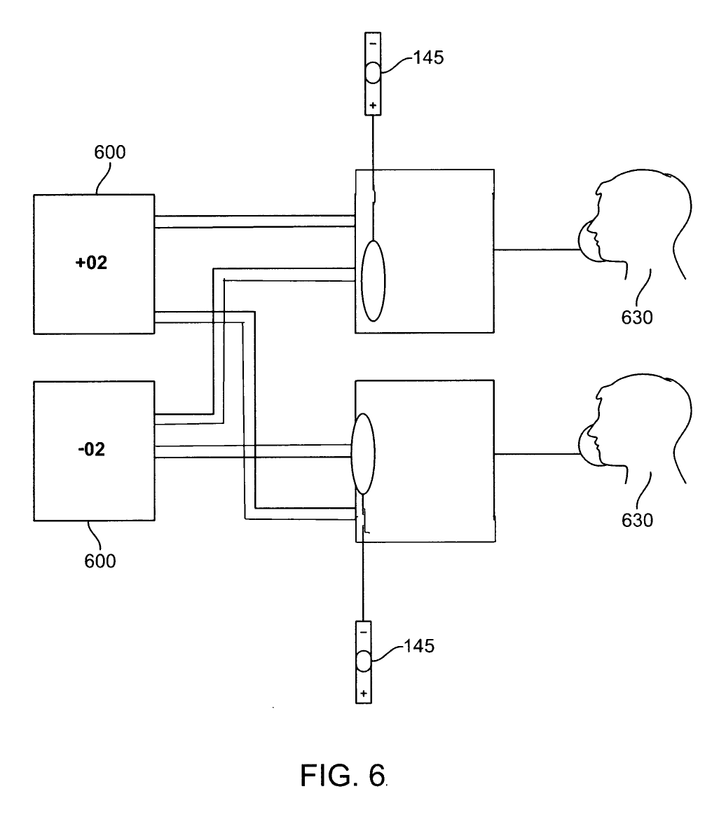

FIG. 7 is an embodiment of the apparatus 700 of the present invention, wherein the air-reservoir 703 can include more than two air chambers 710+, wherein said air chamber can each contain a different type of gas. Each air chamber can be accessed by each user 735 through use of the apparatus switch and control unit 745. All air chambers 710+ are connected to the switch and control unit of the present invention.

The various gases that are suitable for use in the air chambers 710+ can include carbon dioxide, nitric oxide, helium, nitrous oxide. Table 1 is an embodiment of the properties of the gases that can be use in the present invention

TABLE 1
Gases suitable for use in Apparatus

| | | |
|---|---|---|
| Oxygen | O2 | Liquid/gas |
| Carbon dioxide | CO2 | Liquid/gas |
| Carbon monoxide | CO | gas |
| Nitrous oxide | N2O | Liquid/gas |
| Nitric oxide | NO | gas |
| Helium | He | gas |

In one embodiment, as shown in FIG. 7, the air-reservoir 205 of the apparatus 200 can include additional air chambers, wherein the air chambers can store different gases for delivery to the user 230.

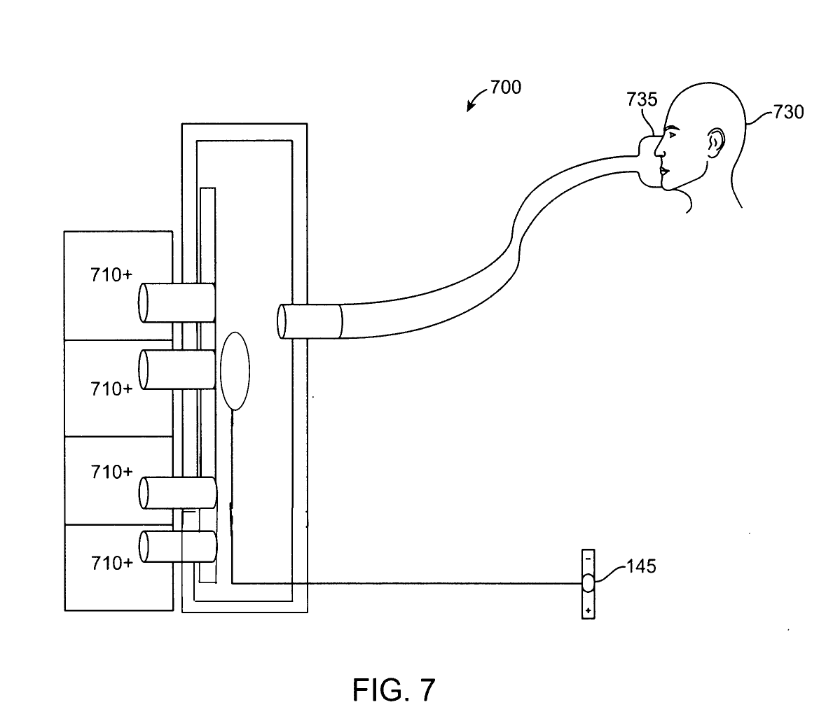

FIG. 8 is an embodiment of the apparatus 800 having a nebulizer 805 to allow for the introduction of medicinal products to the user 830 while using the apparatus 800. Suitable nebulizers 805 can include jet nebulizers.

In one embodiment, supplemental agents such as medicinal products or water can be added to the inhalation air delivered to a user, whereby such supplemental agents can be added by hydrolysis, vaporization, nebulization, and ionization.

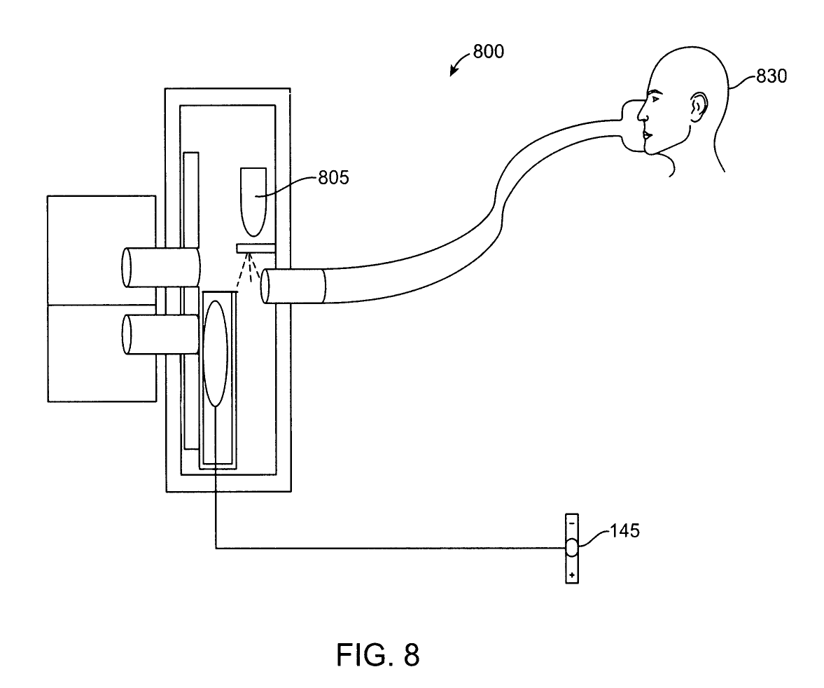

FIG. 9 is an embodiment of the apparatus 900 having a regulator 903 and a flowmeter 901. In such an embodiment, the regulator 903 gives the user 930 the ability to adjust the pressure of the gas flow. The regulator 903 works in conjunction with the flowmeter 901. The flowmeter 901 is housed in the control unit 907 of the invention. The flowmeter 901 reads and measures the flow of the gas being delivered to the user 930.

Suitable regulators 903 for the present invention include single stage regulators, multi-stage regulators, preset regulators, or adjustable regulators. Suitable flowmeters 901 useful for the invention include Thorpe tube flowmeters, burbon flowmeters, or flow restrictors.

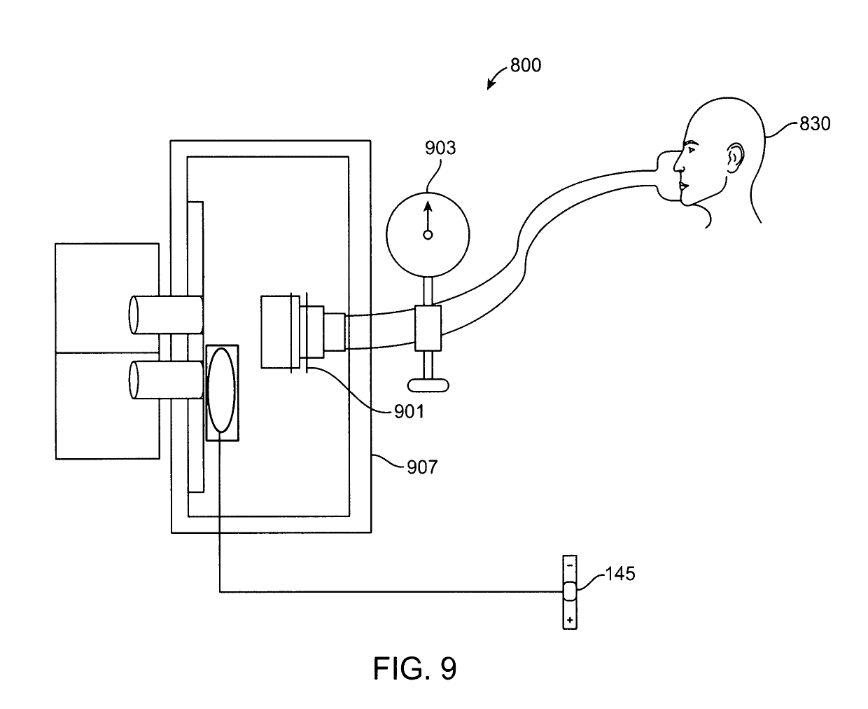

FIG. 10 is an embodiment of the apparatus using a membrane 1000 to equalize pressure throughout the present invention. It is comprised of a membrane 1000, an air separator 1001, a high oxygen compartment 1003, a low oxygen compartment 1005, air ducts 1007, an airflow control means such as a slide gate 1009, and a breathing apparatus 1011.

To explain briefly, the membrane 1000 separates the high concentration compartment 1003 from the low concentration compartment 1005. This embodiment uses a three layer design where the first layer, second layer, and third layer are stacked. The encapsulated between the first layer and second layer comprises the high oxygen compartment. The volume encapsulated between the second and third layer comprises the low oxygen compartment.

In this embodiment, the air separator 1001 produces a high volume of low oxygen concentration air. The second layer serves as a flexible membrane. The membrane enables the low concentration compartment 1005 to fill until the membrane exerts pressure on the high concentration compartment 1003. This causes the pressure in both compartments to equalize.

The addition of a first pressure release valve 1004 in the low concentration compartment 1005 limits the pressure in both compartments to the pressure release setting in the low concentration compartment 1005.

The addition of a second pressure release valve 1002 in the high concentration compartment 1003 enables the oxygen compartment to be pressurized to any value equal or higher than the pressure in the low concentration compartment 1005 without venting oxygen.

This embodiment enables pressure in both the high concentration compartment 1003 and low concentration compartment 1005 to be controlled to overcome air flow resistance in the breathing apparatus comprised of ducts and mask.

The membrane 1000 separates the high concentration oxygen compartment 1003 and the low concentration oxygen compartment 1005. The air separator 1001 takes in ambient air and filters it into the low oxygen compartment 1005. As more low concentration oxygen filters into the low oxygen compartment 1005, the flexible membrane 1000 exerts pressure on the high concentration oxygen compartment 1003, as demonstrated in 1013 (FIG. 10(b)). As more pressure is exerted on the high concentration oxygen compartment, the pressure is equalized 1015. The airflow control means such as a slide gate 1009 gives users their choice of air concentration. The breathing apparatus 1011 houses the air ducts 1007 and the slide gate 1011. The air flows out of whichever duct 1007 the slide gate is not in front of.

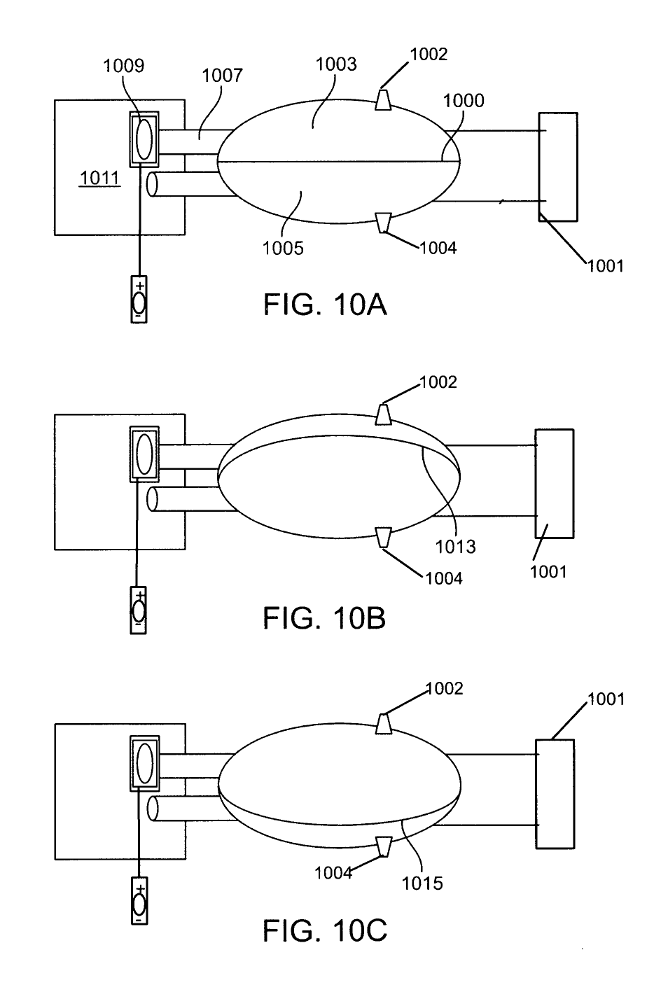

FIG. 11 illustrates the use of an airflow constriction in an embodiment of the apparatus. It is comprised of the air-flow constriction 1100, the high oxygen compartment 1101, the low oxygen compartment 1103, the low oxygen duct 1105 and the high oxygen duct 1107.

As shown in FIGS. 11a and 11b, the airflow constriction 1100 can be either raised to let air flow easily from the high oxygen compartment 1101 or the low oxygen compartment 1103 or lowered to increase airflow resistance.

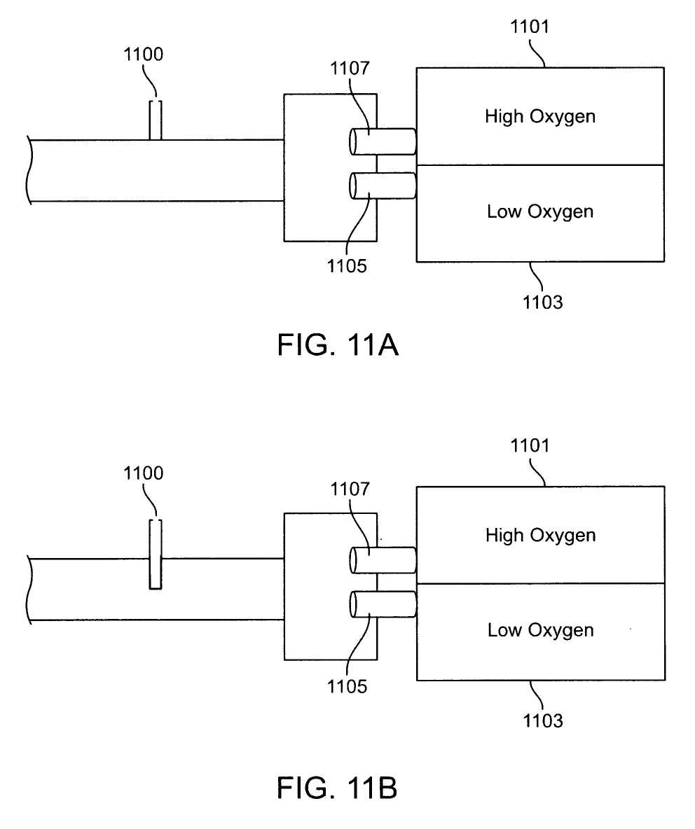

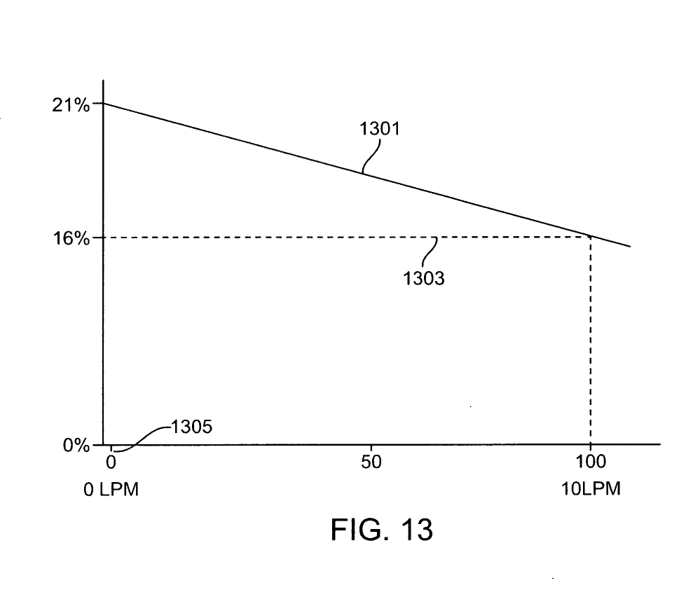

The first means involves limiting the air separation utilizing the variable flow oxygen valve in an air separator as in FIG. 12. The second means involves selecting the lower oxygen concentration filtrate during the partial swing absorption air separation cycle as illustrated in FIG. 14-16.

It will be apparent to those skilled in the art that an embodiment utilizing combined means to control the oxygen concentration in the low oxygen mixture from approximately 8% and 12% to achieve exemplary challenge level for a wide range of users. Embodiments utilizing either or both of these means fall within the scope of this invention.

FIGS. 12A, 12B, and 12C illustrates the air separators including a variable flow oxygen valve that determines the amount of oxygen removed from the filtrate air used as a source low oxygen air in an embodiment of the apparatus. It is comprised of the apparatus 1200, the ambient air 1201, the flow oxygen adjustment 1203, the ambient air duct 1205, and the oxygen filtrate 1207.

Ambient air 1201 flows in from the duct 1205 and is put through the flow oxygen adjustment 1203. As shown in FIG. 12a, the ambient air 1201 can go into the apparatus 1200 without filtration, giving the low oxygen the same concentration as room air. As shown in FIG. 12b, the flow oxygen adjustment 1203 removes the maximum amount of oxygen from the ambient air 1201 and into the filtrate 1207.

In an embodiment, via the switching mechanism, in the event a low concentration oxygen is present in the air-reservoir, the percentage of oxygen removed from the filtrate stream can be controlled, with control occurring by reducing the amount of oxygen the air separator is allowed to filter out. The filtrate stream is that air present in the air-reservoir that will be used in part to form the inhalation-air. In such an embodiment, the air separator includes a variable flow oxygen adjuster, also known as an oxygen flow valve. Reducing the oxygen flow rate increases the fraction of oxygen vented in filtrate. This enables the user to vary the amount of oxygen removed from the filtrate from zero, meaning ambient air, to about 30% by adjusting the oxygen flow valve. This enables a user to set the challenge level resulting from reduced oxygen air from ambient air up to the average oxygen removed in the filtration cycle.

The release rate, normally controlled by a valve, and a control enables the partial pressure of oxygen in the low oxygen concentration compartment to range from ambient air, up to the ambient air taken unless the maximum amount of oxygen the air separator can remove. Usually, this is about 30% oxygen, which can be reduced down to about a minimum of 16% oxygen.

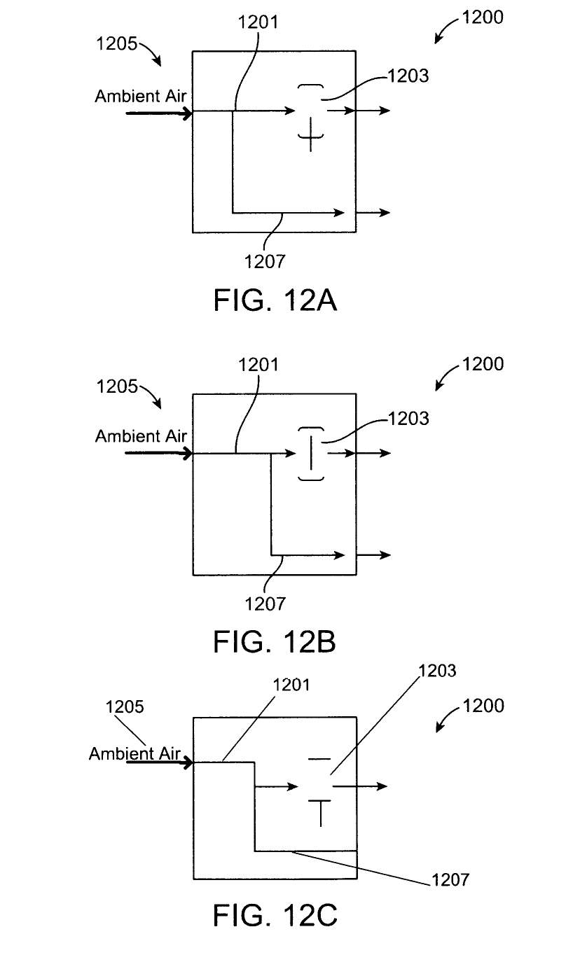

FIG. 13 illustrates a graphical representation of the variable flow oxygen adjustment. The graph is comprised of the amount of oxygen left in the low oxygen concentration left by the flow oxygen adjustment 1301, the minimum amount of oxygen the low oxygen concentration can have 1303, and the oxygen flow percentage 1305. Maximal oxygen flow creates maximal removal of oxygen from the low oxygen air. Reduction in oxygen flow decreases the amount of oxygen removed thus increasing the amount of oxygen in the low oxygen air. At a zero oxygen flow rate, no oxygen is removed from the low oxygen air so at the minimum flow setting the low oxygen air has the same oxygen concentration as the environment. Since the amount of oxygen removed from the air is adjustable, the amount of oxygen in the low oxygen air is adjustable from room air minus the maximum capacity of the air separator equivalent to the oxygen production capacity of the air separator, normally 5, 10 or 15 Liters per minute.

FIG. 14 illustrates the graphical representation of the oxygen filter, the concentration of gases across the filter, and the air near the inlet released during the vent.

As shown in FIG. 14a, ambient air 1400 comes through the oxygen filter. Partial swing adsorption air separators utilize a small pore sieve bed with a pore size to allow O₂ molecules 1403 to pass and with higher resistance to N₂ molecules 1401. As the cylinder is repeatedly pressurized O₂ molecules 1403 are pushed further toward the far end and finally exit as concentrated O₂ 1405. Each cycle vents nitrogen 1401 back to the environment during a blowback release. The blowback cycle varies from 4 to 12 seconds.

As shown in FIG. 14b, the ambient air entering the oxygen is comprised of 78% Nitrogen 1407, 21% Oxygen 1409, and 1% other properties. As the ambient air is put through the oxygen filter repeatedly, the concentration of the air is now 90% Oxygen 1409 and 10% Nitrogen 1407.

As shown in FIG. 14c, during the vent release of blowback air into the environment, Oxygen concentrated air is released and the concentration of Nitrogen 1411 is 18%. As the release cycle progresses, pressure 1415 is used to select the blowback air. As the pressure 1415 is lowered, the oxygen concentration is at a minimum and the nitrogen concentration 1411 goes past its average 1413 and is at its maximum.

The respective percentage of the oxygen in the blowback cycle varies from highest at the beginning to lowest at the end. Air at the entrance to the sieve bed is ambient air, while air deeper in the sieve bed has had more oxygen removed. The oxygen concentration in the blowback release decreases during the blowback cycle.

Pressure during the blowback cycle decreases as the air at the entrance to the sieve bed exits under high pressure, and the air deeper.

In another embodiment, a user is able to select very low oxygen concentration air, lower than average air. This very low oxygen concentration is achieved by venting the higher pressure phase of the filtration cycle. During the early release cycle of the oxygen concentration of the filtrate is approximately proportional to the oxygen concentration of the filtrate. Use of an adjustable pressure release valve that vents the release cycle (not the reservoir compartment), enables retention of the lower oxygen concentration filtrate. This venting enables further reduction of the oxygen partial pressure in the “low oxygen concentration” compartment. Use of this technique enables further reduction of the oxygen concentration down to about 8%. This challenge level enabled by lower oxygen concentration air provides further challenge potential which is beneficial for able-bodied persons to achieve more intense training. Conversely, physically challenged persons with limited athletic capacity, can achieve elevated respiratory challenge levels, which potentiates the effects of oxygen when they switch back.

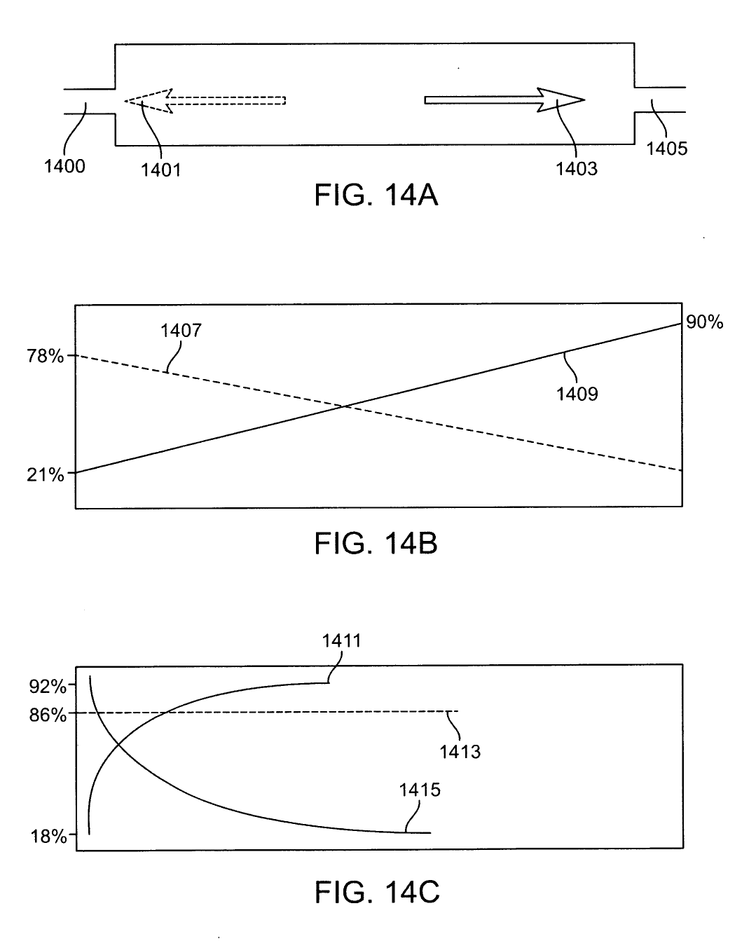

FIG. 15 illustrates the pressure controlled diversion oxygen vent in an embodiment of the apparatus. It is comprised of the low concentration oxygen reservoir 1500, the diversion switch 1501, and the blowback vent 1503. The pressure controlled diversion switch 1501 activates as low oxygen is brought into the low concentrated oxygen reservoir. The blowback of higher concentrated oxygen is sent out of the vent 1503 by the diversion switch 1501.

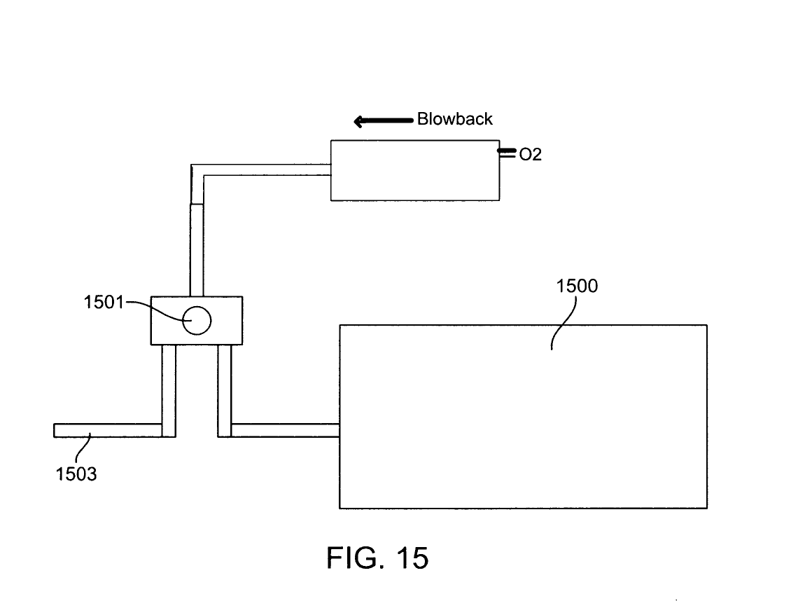

FIG. 16 illustrates an alternative embodiment using compressed gas storage means. It is comprised of an air separator 1600, high oxygen air duct 1601, a low oxygen air duct 1603, compressed low oxygen reservoir 1605, a compressed high oxygen reservoir 1607, low air decompression means 1609, high oxygen air decompression means, high oxygen duct 1613, low oxygen duct 1614.

The air separator 1600 brings in ambient air from the atmosphere. This ambient air is then taken to through the high 1601 and low 1603 oxygen ducts to their respective compression means 1611 and 1612 and, stored in their respective air reservoirs 1605, 1607. This newly compressed air is then sent through the air decompression 1609 and 1611, connected to ducts 1613/1614, connected to air flow control means, FIG. 4, and to the user.

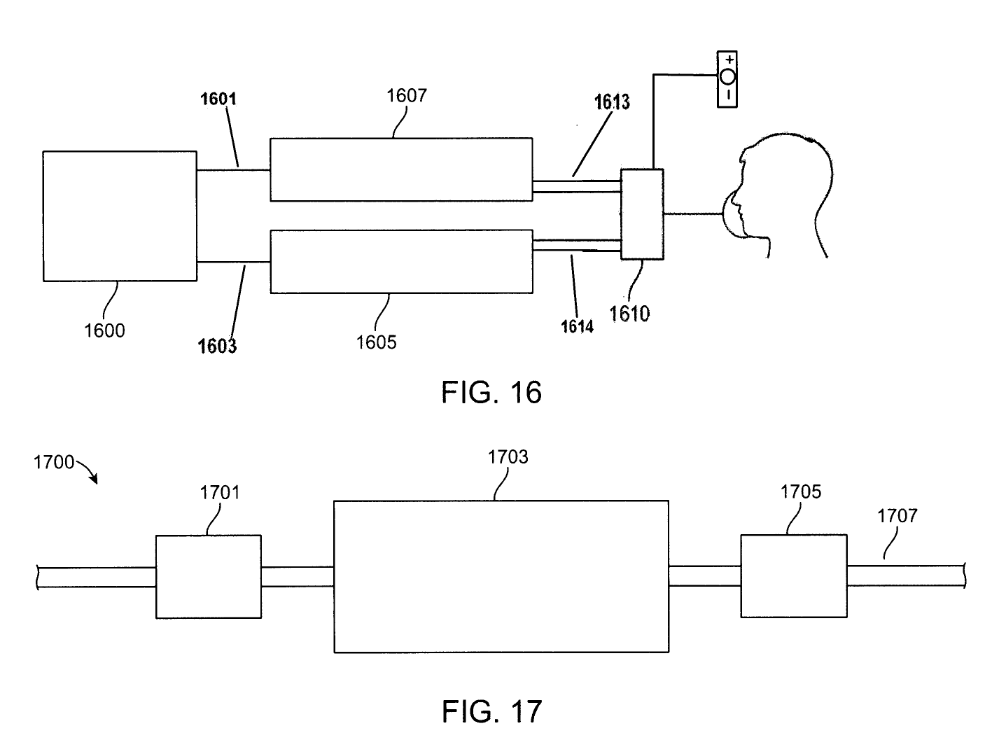

FIG. 17 illustrates the compressed air reservoir in an alternative embodiment of the present invention. It is comprised uncompressed high or low oxygen air 1700, a gas compressor 1701, the high pressure container 1703, a gas regulator 1705, and decompressed air 1707.

The uncompressed air 1700 comes from the air separator to the gas compressor 1701. The gas compressor 1701 senses the presence of input gas activates to compress the gas to a higher pressure in a container 1703 capable of storing pressurized gas. The compressed air is stored in the high pressure container 1703. When the air is needed, it is put through the gas regulator 1705. The gas regulator 1705 allows a pressurized gas to expand to ambient pressure by sending demand and then opening a valve to allow pressurized gas to satisfy the demand. The now ambient-pressured air 1707 is sent to the valve and to the user.

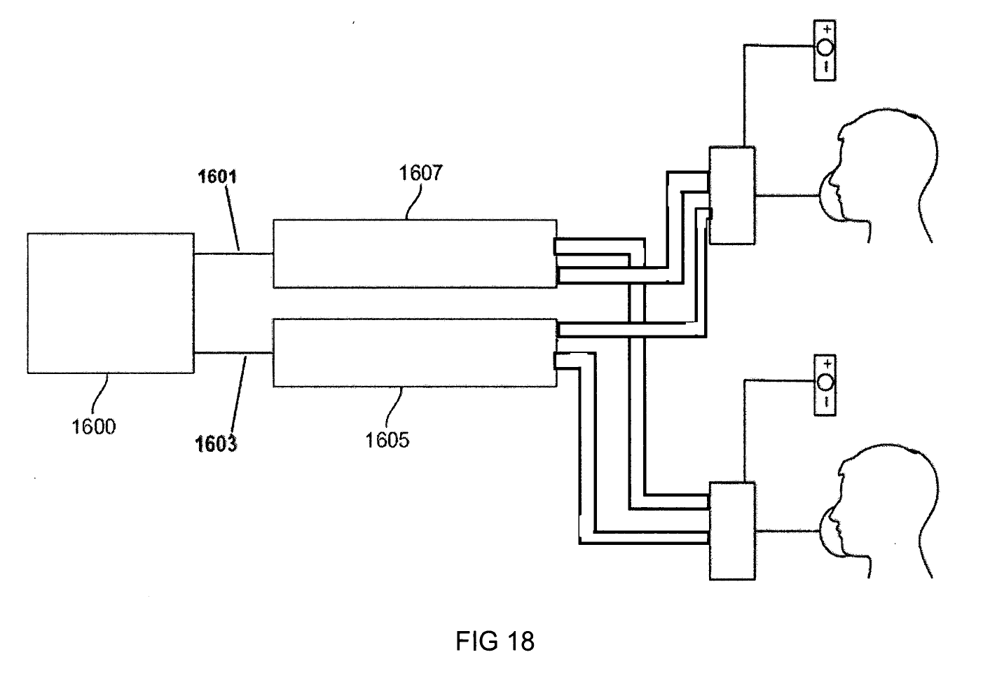

FIG. 18 is an embodiment showing the embodiment of FIG. 17 used for multi-users, alike the invention of FIG. 6.

In a still further embodiment, the physical volume of the reservoir containing uncompressed high oxygen and low oxygen mixtures is too large for certain environments and applications. This is particularly true in mobile field applications, medical, military, industrial, and multi-user environments.

A still further embodiment includes gas compression means for the high oxygen reservoir. A further embodiment includes gas compression means for the low oxygen reservoir.

These embodiments further comprise an input gas compression means, pressurized storage, and an output gas regulator for each the high oxygen reservoir and the low oxygen reservoir. This embodiment enables the physical volume of the embodiment to be reduced proportionality to the compression of stored gasses.

Those skilled in the art will recognize that both air compression and regulators are well-known. A compressor senses presence of input gas activates to compress the gas to a higher pressure in a container capable of storing pressurized gas. A regulator allows a pressurized gas to expand to ambient pressure by sending demand and then opening a valve to allow pressurized gas to satisfy the demand.

Those skilled in the art will also recognize that there are different sensing means for presence of input gas as either volumetric or pressure sensors. A volume sensor uses an expandable compartment which when inflated to a trigger threshold activates a compressor. A pressure sensor senses an increase in input gas pressure to a trigger threshold and activates a compressor.

Those skilled in the art will recognize there are different sensing means for demand for an output gas comprising either volumetric or pressure sensors. A volume sensor uses a collapsible compartment, which when deflated below a threshold value opens a value to allow pressurized gas to refill the compartment. A pressure sensor senses a reduction in pressure below a trigger threshold to open a valve to allow pressurized gas to satisfy demand.

The present invention offers various advantages as it allows switching of the position of the valve to allow release of selective inhalation-air from the air-reservoir. This principle is utilized in restoring the blood flow, accelerate tissue regeneration, improve physical performance, improve fluid intelligence, disease avoidance, disease recovery. The apparatus is useful for athletes to do altitude contrast training.

I claim:

1. An apparatus for altitude contrast training to a plurality of users comprising means to supply switchable high oxygen and low oxygen air mixtures to a plurality of users breathing through a plurality of breathing apparatus.

2. An apparatus according to claim 1 further wherein means to supply switchable high and low oxygen mixtures further comprises:
an air-reservoir to store inhalation-air having:
a first air chamber to store a first concentration of inhalation-air; and
a second air chamber to store a second concentration of inhalation-air; and
two or more breathing apparatus, whereby each user has their own breathing apparatus, to transfer the inhalation-air to the users for facilitating breathing; and
a control unit for each mask to control the flow of inhalation-air from the air-reservoir to each breathing apparatus, the control unit having:
a housing to receive the inhalation-air from the air-reservoir;
plurality of ducts protruding from the housing to connect with the air-reservoir and with the mask, wherein a first duct configured with the first air chamber to supply the first concentration of inhalation-air to the housing, and a second duct configured with the second air chamber to supply the second concentration of inhalation-air to the housing, and a third duct to transfer the received inhalation-air by the housing from the air-reservoir to the mask; and
at least one valve configured to control the flow of inhalation-air from the first duct and the second duct to the housing; and
a switch unit positions the valve to selectively open and close the first duct and the second duct for regulating the flow of inhalation-air from the air-reservoir to the housing; and
one or more tubular conduits attached to the third duct to transfer inhalation air from the housing to the mask.

3. The apparatus according to claim 2 further comprising plurality of filter units attached to each duct to filter the inhalation-air passing to the users.

4. The apparatus according to claim 2, further comprising at least two switch units, wherein each mask has associated with it at least one switch unit.

5. The apparatus according to claim 2, wherein the switch unit further comprises:
a cable that positions the valve; and
a mechanical switch having a first position that allows the housing to receive the inhalation air from the first chamber and a second position that allows the housing to receive the inhalation air from the second chamber.

6. The apparatus according to claim 2 where the switch units are controlled together so that each user receives the same air mixture from the reservoir.

7. The apparatus according to claim 1 where each breathing apparatus is a mask.

8. The apparatus according to claim 2, where the control unit further comprises an underflow valve to automatically to ambient air when the reservoir becomes empty.

9. An apparatus according to claim 1 further wherein means to supply high and low oxygen mixtures further comprises:
a source of high oxygen air connected to an air compression means, and a compressed high oxygen air storage means; and
a source of reduced oxygen air connected to an air compression means and a compressed low oxygen air storage means.

10. An apparatus according to claim 9 wherein means to supply high and low oxygen mixtures further comprises:
a means to extract and decompress high oxygen air from compressed high oxygen storage means; and
a means to extract and decompress low oxygen air from compressed low oxygen is storage means; and
a control means that controls delivery of high oxygen air or low oxygen air to each of the plurality breathing apparatus.

11. An apparatus according to claim 10 wherein control means is a plurality of valves that controls delivery of high oxygen or low oxygen air to each of the plurality of breathing apparatus.

12. An apparatus according to claim 10 wherein each of the plurality of valves is controlled by the user breathing through each breathing apparatus.

13. An apparatus according to claim 11 wherein the control means is a single control that controls delivery of high oxygen or low oxygen air to the plurality of breathing apparatus.

14. An apparatus according to claim 10 wherein the control means is a single valve that controls the delivery of the high oxygen air or low oxygen air to the plurality of breathing apparatus.

15. An apparatus according to claim 1, wherein the plurality comprises a single user and a single breathing apparatus.

16. An apparatus according to claim 10, further comprising underflow protection means wherein depletion of air from the high or low oxygen storage means supplies environmental air to the breathing apparatus.

17. An apparatus according to claim 16, wherein control means further comprises underflow protection means that supplies environmental air to the breathing apparatus upon depletion of compressed high oxygen or low oxygen air in the storage means.

18. An apparatus for altitude contrast training with variable pressure to a user comprising means to supply adjustable positive and negative pressure inhalation air to a user, altitude contrast training comprising means to supply switchable high oxygen and low oxygen air mixtures to a user breathing through a breathing apparatus.

19. An apparatus according to claim 18 further comprising variable positive pressure:
an air-reservoir to store inhalation-air having:
a first air chamber to store a first concentration of inhalation-air with a pressure release means; and
a second air chamber to store a second concentration of inhalation-air with a pressure release means;
a flexible membrane separating first air chamber and second air chamber to equalize pressure between the first and second air chambers;
a control unit to control the flow of inhalation-air from the air chambers of the air reservoir to a breathing apparatus.

20. An apparatus according to claim 19 control unit further comprising,
a housing to receive the inhalation-air from the air-reservoir;
plurality of ducts protruding from the housing to connect the air-reservoir with the mask, wherein a first duct configured with the first air chamber to supply the first concentration of inhalation-air to the housing, and a second duct configured with the second air chamber to supply the second concentration of inhalation-air to the housing, and a third duct to transfer the received inhalation-air by the housing from the air-reservoir to the breathing apparatus; and
a valve configured to control the flow of inhalation-air from the first duct and the second duct to the housing; and
a switch unit positions the valve to selectively open and close the first duct and the second duct for regulating the flow of inhalation-air from the air-reservoir to the housing; and
one or more tubular conduits attached to the third duct to transfer inhalation air from the housing to the breathing apparatus.
a switch unit positions the valve to selectively open and close the first duct and the second duct for regulating the flow of inhalation-air from the air-reservoir to the membrane; and
a slide gate to toggle between said two outgoing air ducts.

21. The apparatus for altitude contrast training in claim 19, wherein said first chamber contains high oxygen air and second chambers contains low oxygen air.

22. The apparatus for altitude contrast training in claim 19, wherein said breathing apparatus is a mask.

23. The apparatus for altitude contrast training in claim 19, wherein said breathing apparatus is two or more masks to allow use by two or more users.

24. The apparatus in claim 19, further comprising an negative pressure means as an airflow constriction.

25. The apparatus in claim 19, further comprising a negative pressure means as a fan operating to oppose airflow to the breathing apparatus.

26. The apparatus in claim 19, further comprising a positive pressure means as a fan operating to aid airflow to the breathing apparatus.

27. The apparatus in claim 19, wherein pressure release means of the first chamber controls the maximum positive pressure allowed in the first chamber when the first chamber is fully inflated to avoid over-inflation.

28. The apparatus in claim 19, wherein pressure release means of the second chamber controls the positive pressure delivered to the breathing apparatus at all times when the first chamber is not fully inflated.

29. The apparatus in claim 19, wherein pressure release means of the first chamber is set equal to or slightly higher than the pressure release means of the second chamber.

30. An apparatus for altitude contrast training comprising means to deliver switchable high oxygen and low oxygen air with additional means to vary the oxygen partial pressure of low oxygen air to a user breathing through a breathing apparatus.

31. The apparatus in claim 30, wherein means to vary the oxygen partial pressure of low oxygen air from from about 21% to about 16% comprises restriction of oxygen extraction in the air separation process by restriction of the variable flow oxygen valve in an air separator.

32. The apparatus in claim 30, wherein variable low oxygen air mixture comprises means to vary oxygen concentration in low oxygen air from about 8% to about 16% by selecting the lower oxygen air from the later phase of the partial swing absorption process.

33. The apparatus in claim 30, comprising a means to control the oxygen partial pressure of between approximately 8% and 21% utilizing a plurality of means of to control oxygen partial pressure in low oxygen air.

34. The apparatus in claim 33, wherein the plurality of means comprises restriction of oxygen extraction and selection of the low oxygen air from the later phase of the partial swing absorption process.

35. An apparatus for altitude contrast training comprising means to supply switchable high oxygen and low oxygen air mixtures to a user breathing through a breathing apparatus further comprising means to supply supplemental agents to a user by mixing the supplemental agent in the switchable high or low oxygen air mixture delivered to the user through the breathing apparatus.

36. The apparatus of claim 35, wherein means to supply supplemental agent comprises nebulization of a medicinal or health supportive substance miscible in water, wherein the supplemental agent is mixed with the switchable air supply delivered to the user through the breathing apparatus.

37. The apparatus of claim 35, wherein means to supply supplemental agent comprises addition of a medicinal or health supportive gas, wherein the supplemental gas is mixed with the switchable air supply delivered to the user through the breathing apparatus.

38. The apparatus of claim 35, wherein means to supply supplemental agent comprises addition of a medicinal or health supportive vapor produced by evaporation of a substance with the switchable air supply delivered to the user through the breathing apparatus.

39. The apparatus of claim 35, wherein means to supply supplemental agent comprises addition of a medicinal or health supportive hydrolysis product like hydrogen where the substance is produced by hydrolysis of a substance with the switchable air supply delivered to the user through the breathing apparatus.

40. The apparatus of claim 35, wherein means to addition of the supplemental agent is supplied during the low oxygen phase the agent has high oxygen reactivity.

41. The apparatus of claim 35, wherein means to addition of the supplemental agent is supplied during the high oxygen phase the agent's effect is improved by high oxygen.

42. The apparatus of claim 36, wherein the supplemental agent comprises glutathione or any antioxidant, colloidal silver, or any other antimicrobial agent miscible in water, or homeopathic remedy.

43. The apparatus of claim 37, wherein the supplemental agent comprises nitrous oxide, carbon dioxide, hydrogen, or any other gas known to have a medicinal or health promoting effect.

44. The apparatus of claim 38, wherein the supplemental agent comprises any volatile essential oil including but limited to tea tree oil, peppermint oil, eucalyptus oil, camphor or any other essential oil having medicinal or health promoting effect.

45. The apparatus of claim 39, wherein the supplemental agent comprises water broken down by hydrolysis, wherein hydrogen is a electrolytic product.

46. An apparatus for altitude contrast training comprising means to supply switchable high oxygen air and environmental air comprising a reservoir containing a high oxygen air, an air supply switching means, a user control means, and a breathing apparatus,
the reservoir comprising an inflatable reservoir made of oxygen impermeable material that does not give off chemicals; and closeable port with dimensions of sufficient size for inspection, cleaning and rapid drainage;
the air supply switching means comprising a duct connected to the inflatable reservoir, a second duct open to environmental air, and a third duct connected to a conduit connected the breathing apparatus; and a switch mechanism that permits air to flow from the environment or from the reservoir but not both; and
user control means enabling the user to control airflow through the switching means from the high oxygen reservoir or environmental air; and
a breathing apparatus comprising a mask.

47. The apparatus of claim 2, wherein the first air chamber and second air chamber further comprise a closeable port with dimensions of sufficient size for inspection cleaning and rapid drainage.

48. The apparatus of claim 20, further comprising an oxygen level sensor in the conduit connected to the breathing apparatus.
